(19) United States
(12) Patent Application Publication
(10) Pub. No.: US 2006/0063972 A1
(43) Pub. Date: Mar. 23, 2006

(54) Closure mechanism for an endoscopic overtube having a slot

(76) Inventor: Stanley F. Chang, Fresno, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 10/947,629

(22) Filed: Sep. 21, 2004

(51) Int. Cl. A61B 1/00

(52) U.S. Cl. 600/114

(57) ABSTRACT

A locking mechanism for an endoscopic overtube comprising a tubular member having a slot extending from the distal end to the proximal end of the tubular member for placement of an endoscope into the lumen configured to slidably receive the endoscope. The slot is cut through a thickened portion that forms a ridge having a plurality of generally transverse or angular holes on opposite sides of the slot. In one embodiment, the overtube is loaded onto the endoscope and then the slot is securely closed by threading an elongated member, such as a string or string-like member, through the holes and pulling the elongated member taut. In another embodiment, the overtube is pre-threaded with the elongated member forming a plurality of loops configured to go over the endoscope and components attached thereto. Once the endoscope is loaded, the elongated member is pulled taut to securely close the slot.

### BACKGROUND OF THE INVENTION

1. Field of the Invention

The field of the present invention relates to a closure mechanism to maintain three-dimensional closure of a longitudinal slot in an endoscopic overtube, such as the type utilized in gastrointestinal endoscopy, particularly colonoscopy. More specifically, the invention described herein is an improved closure mechanism to securely and safely close the slot so as to facilitate use of the overtube for such procedures.

2. Background

As used herein, the terms “splint”, “splinting device”, and “overtube” are used interchangeably to refer to a generally elongated hollow tubular member that is adaptable for receiving a medical instrument, such as an endoscope. The term “endoscope” or “scope” is used to refer to a colonoscope, gastroscope, enteroscope, or other types of medical endoscopes. An endoscope generally consists of a connecting tube, a handle and an insertion tube (the part inserted into the patient). In referring to the opposite ends of the splint or scope, the “proximal end” refers to that part of the splint or scope which is closest to the hands of the operator or physician endoscopist performing the procedure, and the “distal end” refers to that part of the splint or scope farthest from the operator or, physician endoscopist (hereinafter collectively referred to as “operator”).

In gastrointestinal endoscopy, especially colonoscopy, straightness of the endoscope is necessary, or at least desirable, for advancement of the endoscope. Colonoscopy is the most sensitive and specific means for examining the colon, particularly for the diagnosis of colon cancers and polyps. Because the cecum, the portion of the colon furthest from the anus, can be a common location for cancer, it is generally desirable that the entire colon be completely examined. During a colonoscopy, the scope is inserted into the anus, through the rectum, sigmoid colon, descending colon, transverse colon, ascending colon and then into the cecum. Advancing the scope, which is typically about 160 centimeters in length, can be difficult due to a loop in the sigmoid colon. As is known to those skilled in the art, failure to substantially straighten the loop in the sigmoid colon prior to continuing can cause enlargement of the loop and difficulty in advancement of the scope. It can also result in pain and damage, including adverse cardiac reactions such as hypotension and bradycardia. Therefore, during the typical endoscopic procedure, after the operator reaches the descending colon or the transverse colon, he or she reduces and straightens the sigmoid loop by withdrawing the scope to a generally straightened position. Once the loop is straightened, further advancement of the endoscope can usually be accomplished. Unfortunately, it is not uncommon that upon readvancement of the scope, the sigmoid loop has a tendency to reform. Maneuvers to prevent or limit reformation of the sigmoid loop include abdominal compression and changing the position of the patient. However, sometimes these maneuvers are ineffective.

A sigmoid splint or overtube is useful in preventing reformation of the sigmoid loop to facilitate advancement of the scope. As is well known, however, when the need for a sigmoid splint arises, the tip of the scope is usually already in the proximal colon. At this point, the operator may opt to remove the scope entirely in order to load the endoscope inside the sigmoid splint, in an end-to-end fashion, and start the procedure over again. A more convenient way, however, is to be able to load the endoscope into the splint without having to remove the scope from the patient. This is done by using a splint with a longitudinal slot along the length of the splint. The slotted splint is loaded onto the endoscope in a side-to-side fashion by opening the slot, such that the scope does not have to be removed from the patient. Once placed around the scope, the slot is closed. The slot will then need to be fastened or locked in the closed position. A fastening or locking mechanism is needed to keep the splint closed in a secure fashion.

Another area of the gastrointestinal tract where medical problems are known to exist and treatment can benefit from use of an endoscope are arterio-venous malformations or other lesions located in the small bowel, which can be a cause of gastrointestinal bleeding. Utilizing endoscopes to investigate and treat these problems, by inserting a scope through the patient's mouth, past the stomach and into the small bowel, however, is very difficult to accomplish due to the fact that the scope tends to form a loop in the stomach. The looping of the endoscope in the stomach, which swells the stomach, makes passage deeply into the small bowel generally difficult with standard scopes. If a scope can reach deeply into the small bowel, then more lesions will be detected and treated.

3. Related Art

A splinting tube with a straight longitudinal slot, which enables side-to-side loading onto the scope, is available (Olympus America, Inc., Melville, N.Y.). The slot is a simple straight opening along the length of the splint. Once loaded onto the colonscope, the slot is kept closed with adhesive tape. However, this fastening method is difficult because (a) the adhesive tape, or part of it, may come off, especially when, as usual, there is lubricant on the splint; and (b) even if the slot is securely taped, the tape fastens the slot only in two dimensions, such that movement of the two edges against one another can still occur. Another type of closure mechanism that has been disclosed is a zip-locking mechanism (i.e., similar to that used on plastic sandwich bags). However, the process of building a zip-locking mechanism into the wall of the splint is technically difficult, and the closure in the presence of lubricants still may not be very secure. Furthermore, if any bending of the splint occurs, and especially in the presence of lubricants, parts of the zip lock, along this approximately 40 cm length, can come apart.

In U.S. Pat. No. 5,941,815 to Chang (the same inventor as of the present invention), the inventor describes a fastening mechanism using studs built onto one side of the slot, and receiving sockets on the other side of the slot. Closure of the slot is obtained by snapping the studs into their corresponding sockets. This fastening mechanism closes the slot in three dimensions. In the presence of lubricants, however, the fastened studs may be able to separate from the sockets. Furthermore, it is believed that the manufacturing process for this closure mechanism is very expensive. In U.S. Pat. No. 6,712,755, also to Chang, the inventor teaches a locking mechanism wherein a slot is cut in a step-wave type of configuration creating a series of interdigitating members. A small lumen is pre-extruded through these members. Upon closure of the slot, with the members joined together, the lumen becomes continuous and a string is threaded through this lumen, locking the members together to close the slot. This closure mechanism achieves a three-dimensional closure and alignment of the slot. The full disclosures of U.S. Pat. Nos. 5,941,815 and 6,712,755 are incorporated herein by this reference.

What is needed is an improved securing mechanism for closing the longitudinal slot on splints used as an overtube for endoscopic procedures. Such an improved securing mechanism should provide a three-dimensional closure and alignment of the slot to securely close the longitudinal slot. In addition, an improved securing mechanism should be substantially unaffected by the presence of the lubricants and bodily fluids encountered in gastrointestinal endoscopy. Ideally, such an improved securing mechanism should minimize the amount of operator labor and patient discomfort that may be associated with utilizing a splint during an endoscopic procedure, such as a colonoscopy.

### SUMMARY OF THE INVENTION

The closure mechanism for an endoscopic overtube of the present invention provides the benefits and solves the problems identified above. That is to say, the present invention discloses a highly secure closure mechanism for splints having a longitudinal slot that provides three-dimensional closure and alignment of the slot. The closure effect of the present invention is not affected by the lubricants and bodily fluids that are present in endoscopic procedures. The closure mechanism of the present invention simplifies use of the splint during endoscopic procedures and reduces the labor required for those procedures and the likely discomfort of the patient. Specifically, the closure mechanism and method of the present invention is no more cumbersome than the present method of utilizing adhesive tape, but is more secure. The presence of a hydrophilic coating, often beneficially utilized in endoscopic procedures and a problem for effective closure by adhesive tape, does not limit the slot closure ability of the present invention.

In one embodiment of the present invention, the closure mechanism for a splint comprises an overtube made of an elongated cylindrical or tubular member having a proximal end, a distal end and an outer surface. The tubular member is configured with a cross-section having a thickened portion through which a slot is disposed to allow insertion of an endoscope into an endoscope lumen in the tubular member. The thickened portion is provided with a plurality of holes that can be transversely or angularly disposed on opposite sides of the slot. An elongated member, such as a string or string-like member, is threadably received in the plurality of holes and then pulled taut to close the slot and maintain the slot in a closed position. In the preferred embodiment, the elongated member has a trailing end with knot member that cannot pass through the holes and a leading end that is suitable for a fastener member, such as tying it into a knot or providing a separate fastener. A blunt needle or other instrument can be utilized to thread the elongated member through the holes. In another embodiment, the elongated member is formed into a plurality of loops that extend generally outwardly from the overtube.

To perform a gastrointestinal endoscopy procedure in a gastrointestinal tract using an endoscope and an overtube having a longitudinal slot and a closure mechanism according to one embodiment of the present invention, the operator introduces the endoscope into the gastrointestinal tract and continues inserting the endoscope until a loop in the gastrointestinal tract or the endoscope substantially prevents further insertion of the endoscope into the endoscopic tract. At that time, the operator withdraws the endoscope as necessary so as to straighten the loop in the gastrointestinal tract or the endoscope and loads the overtube onto the endoscope by placing the endoscope through the slot into the endoscopic lumen in the endoscope. An elongated member, such as a string or string-like member, is then threaded through a plurality of holes that are disposed in a thickened portion of the overtube, through which the slot is located, by crossing the slot with the elongated member. The slot is closed by pulling the elongated member taut and then maintained in the closed condition by tying or otherwise securing the elongated member. The overtube is then inserted into the gastrointestinal tract and the endoscopy procedure is completed. In the pre-threaded embodiment of the present invention, the elongated member is pre-threaded through the plurality of holes and formed into a plurality of loops extending generally outwardly from the overtube. After withdrawing the endoscope to straighten the loop, the endoscope handle and connecting tube (the so called “umbilical cord”) are separated from the light source, suction tube and water tube so the loops can be placed over the connecting tube, handle and endoscope insertion tube and the insertion tube placed through the slot into the endoscopic lumen of the overtube. The elongated member is pulled taut to securely close the slot and the overtube is inserted into the gastrointestinal tract to complete the endoscopic procedure.

Accordingly, the primary objective of the present invention is to provide a closure mechanism for an endoscopic overtube having a slot with the features generally described above and more specifically described below in the detailed description.

It is also an important objective of the present invention to provide a closure mechanism for a slotted endoscopic overtube that securely closes the longitudinal slot on the overtube to facilitate use of the overtube in endoscopic procedures.

It is also an important objective of the present invention to provide a closure mechanism for an endoscopic overtube that comprises a slot having a thickened cross-sectional portion where the slot is located with a plurality of holes passing transversely or angularly through the thickened portion that is threaded or laced to join together the edges of the slot so as to securely close the slot.

It is also an important objective of the present invention to provide a closure mechanism for an endoscopic overtube having a plurality of holes through a thickened portion of the overtube that are configured to receive a string led by a blunt needle to securely close the slot.

It is also an important objective of the present invention to provide a method of performing gastrointestinal endoscopy using an endoscopic overtube having a slot that utilizes a string threaded through a plurality of holes that are transversely or angularly positioned through a thickened portion of the overtube to securely close the slot.

The above and other objectives of the present invention are explained in greater detail by reference to the attached figures and description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a distal end perspective view of a splint utilizing the closure mechanism of the present invention showing the slot threadably closed by the elongated member;

FIG. 2 is a cross-sectional view of the tubular member showing a hole through the thickened portion;

FIG. 3 is a distal end perspective view showing the slot open and ready to receive an endoscope in the endoscopic lumen thereof;

FIG. 4 is a top view of a splint configured with the closure mechanism of the present invention showing an alternative method of threading the elongated member (i.e., string) through the plurality of holes;

FIG. 5 is a top view of a splint configured with the closure mechanism of the present invention showing another alternative method of threading the elongated member (i.e., string) through the plurality of holes; and

FIG. 6 is a side view of an embodiment of the splint of the present invention having a pre-threaded elongated member formed into a plurality of large loops to be placed over the endoscopic equipment.

### DETAILED DESCRIPTION OF THE PREFERRED EMBODIMENTS

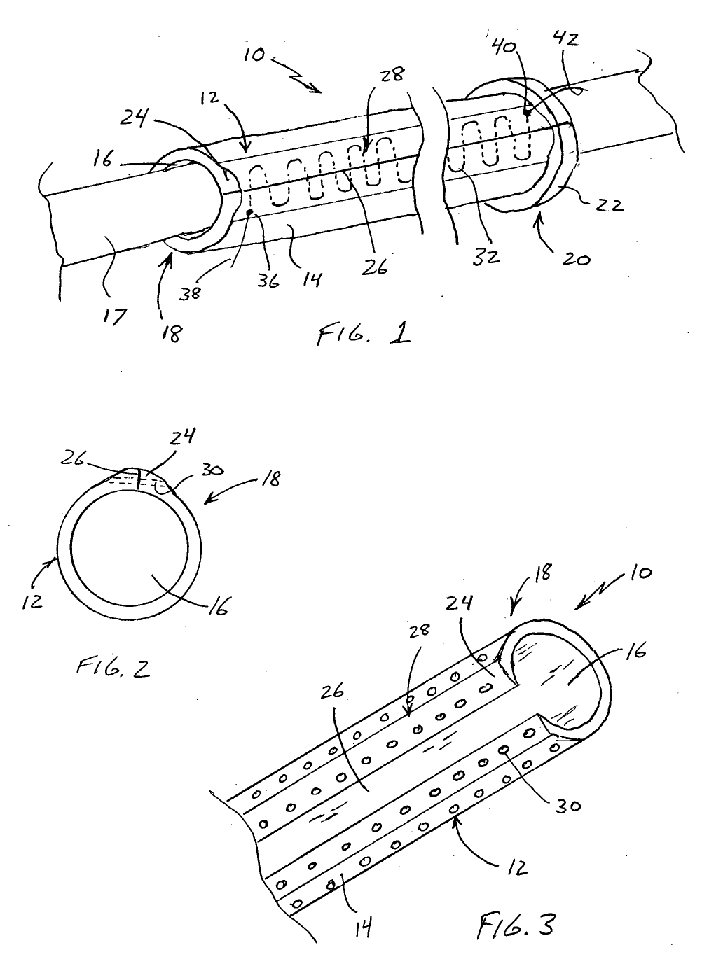

With reference to the figures where like elements have been given like numerical designations to facilitate understanding the present invention, and particularly with reference to the embodiments of the present invention illustrated in FIGS. 1 through 6, the slot closure mechanism of the present invention is suitable for use with an endoscopic overtube, designated generally as 10. The typical overtube 10 includes an elongated tubular member 12 with an outer surface 14 and an endoscope lumen 16 sized and configured to slidably receive the insertion tube 17 of an endoscope 19 therein. Typically, the inside diameter of overtube's endoscope lumen 16 is only slightly larger than the outside diameter of insertion tube 17 to minimize the size of tubular member 12. Tubular member 12 has a distal end 18 which enters the human body and a proximal end 20 which can have an end section 22 having an outside diameter larger than the outside diameter of distal end 18 to prevent complete entry into the human body during the procedures described herein. Tubular member 12 can be shapes other than circular, for instance member 12 can be oval or any other shape that permits easy entry into and passage through the colon. The stiffness of splint 10 can vary along its length. For example, the distal end 18 can be more flexible than the remaining portion of the splint to facilitate easy insertion of overtube 10 into the human colon. Outer surface 14 of tubular member 12 can include a plurality of insertion markings (not shown) at spaced apart intervals to indicate the depth of insertion of tubular member 12 into the colon.

Overtube 10 can be made out of a variety of materials, including rubber, plastic, silicone and others (preferably a relatively soft material that will not damage insertion tube 17 or the colon). Many of the preferred materials can be formed into elongated tubular member 12 by use of an extrusion process. This same process, which forms endoscope lumen 16, can also be used to form a thickened portion 24, best shown in FIGS. 1 and 2, that extends from distal end 18 to proximal end 20. Thickened portion 24 can be formed like a ridge that, as shown in the cross-section of FIG. 2, forms a gentle angle with the cylindrical part of tubular member 12, resulting in a roughly elliptical cross section. In addition to forming the improved closure mechanism, shown generally as 28, of the present invention, thickened portion 24 will provide some additional rigidity to overtube 10. During the manufacturing process, a longitudinally oriented open-ended slot, shown as 26, is cut through thickened portion 24 from distal end 18 to proximal end 20 of tubular member 12. As is known in the art, slot 26 must be sized and configured to removably receive insertion tube 17 into endoscope lumen 16. Using slot 26, insertion tube 17 does not have to be inserted or pre-loaded onto overtube 10 prior to insertion into the patient. In use, after the insertion tube 17 is introduced into the descending colon or transverse colon of the patient, it is straightened in the usual fashion by reducing the sigmoid loop. Overtube 10 is then loaded onto endoscope 19 by opening slot 26 and placing insertion tube 17 inside lumen 16. In the typical prior art, slot 26 is closed by sealing it with adhesive tape on outer surface 14 of tubular member 12. As set forth below, the improved closure mechanism, shown generally as 28, is utilized to provide a more secure and safer closure of slot 26.

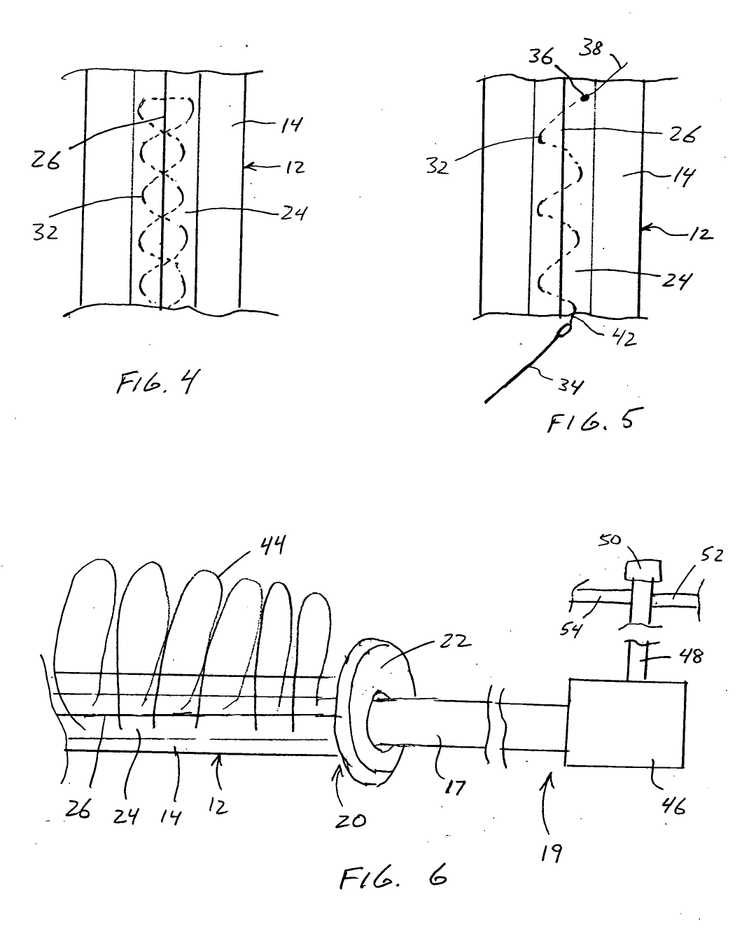

In the preferred embodiment of overtube 10 of the present invention, thickened portion 24 and slot 26 are cooperatively configured to accomplish the objectives set forth herein by providing a plurality of holes 30 transversely or angularly through thickened portion 24 and across slot 26. Holes 30 can be punched through the ridge formed by thickened portion 24 during the manufacturing process, generally before tubular member 12 is cut to form slot 26 therein. As will be obvious to those skilled in the art, holes 30 should be spaced apart a distance sufficient to facilitate secure closure of slot 26 but not so close so as to require an undue amount of effort to obtain that result (i.e., making the closure procedure excessively or unnecessarily cumbersome). In the preferred embodiment, each hole 30 will have at least one other hole 30 that is generally aligned therewith to provide a substantially continuous path through the plurality of holes 30. The path of holes 30 across slot 26 can be generally perpendicular to thickened portion 24 or slot 26, as shown in FIG. 1, or they can be slightly or somewhat angulated in one or more directions relative to slot 26, as shown in FIGS. 4 and 5. Holes 30 should be sized and configured to receive elongated member 32 therethrough. In the preferred embodiment, holes 30 are sized and configured to receive a blunt end needle 34 attached to one end of elongated member 32 and utilized to guide elongated member 32 through the plurality of holes 30. In the preferred embodiment of the present invention, elongated member 32 is a string or string-like member. Alternatively, elongated member 32 can be a wire or the like that is sufficiently flexible and strong so as to pass through holes 30 and securely close slot 26. Also in the preferred configuration, elongated member 32 can have a knot, bulbous or button-like member 36 at trailing end 38 that cannot pass into or through holes 30 once elongated member 32 is pulled taut. A fastener member 40 can be made (i.e., knotted) or supplied at or near leading end 42 at the opposite end of elongated member 32 from trailing end 38 to hold elongated member 32 taut and slot 26 closed. Alternatively, fastener member 40 can be a separate member that is placed on elongated member 32 once it exits the last series of holes 30.

Elongated member 32 can be threaded through holes 30 in a variety of different ways, depending primarily on the configuration of holes 30 on opposite sides of slot 26. As shown in FIG. 1, elongated member 32 can be threaded in a step-wave manner by starting at one end, such as distal end 18 shown, and consecutively threading through the plurality of holes 30 to proximal end 20. In this configuration, needle 34 at leading end 42 is threaded first through holes 30 near distal end 18 and then through the remaining holes 30 in a step-wave manner until needle 34 and leading end 42 exit holes 30 near proximal end 20. When elongated member 32 is threaded through holes 30, it is pulled tight such that knot member 38 is pulled against the first hole 30 and the open sides of slot 26 are brought together to close slot 26. Once slot 26 is tightly closed, a knot 40 can be made at the proximal end 20 to secure slot 26 closed. Alternatively, a separate fastener member 40 can be attached to leading end 42 to secure slot 26 in the closed condition. In the configuration shown in FIG. 4, elongated member 32 is threaded through a pair of holes 30 at either the distal 18 or proximal end 20 of overtube 10 (preferably distal end 18) and both trailing end 38 and leading end 42 are threaded through holes 30 in a cris-cross manner (i.e., similarly to shoelace tying) to the opposite end of overtube 20 where the ends 38 and 42 are tied off or otherwise connected to securely close slot 26 by pulling elongated member 32 taut. As shown in FIG. 4, a needle 34 can be attached to both ends 38 and 42 to assist in the threading of elongated member 32 through holes 30. FIG. 5 illustrates an embodiment where holes 30 are in an angulated relationship relative to thickened portion 24 and slot 26 and the threading of elongated member 32 begins at one end of tubular member 12. In any configuration of threading elongated member 32 through holes 30, the objective is to securely close slot 26 in a three-dimensional manner around insertion tube 17 to assist with the passage of insertion tube 17 through the human body. Although this is preferably accomplished with a single elongated member 32, to reduce the complexity and inconvenience associated with closing slot 26, those skilled in the art will recognize that two or more elongated members 32 could also be utilized to securely close slot 26.

In an alternative embodiment, shown in FIG. 6, overtube 10 is supplied to the operator of endoscope 19 in a pre-threaded condition so all that needs to be done to securely close slot 26 is to pull elongated member 32 taut and tie off the end or ends of elongated member 32. One way to accomplish the objective of this embodiment is to form elongated member 32 into a plurality of loops 44 that extend generally outwardly from tubular member 12 so that overtube 10 can accommodate the various components associated with endoscope 19. As known to those skilled in the art, during use the typical endoscope 19 is connected to a handle 46 for control of insertion tube 17, which is connected to a connecting tube 48 that interconnects handle 46 with a light source 50, suction tube 52 and water tube 54, as shown in FIG. 6. Although handle 46 and connecting tube 48 are typically fixed to insertion tube 17, light source 50, suction tube 52 and water tube 54 usually can be disconnected from connecting tube 48. Loops 44 should be such that overtube 10 can be placed over connecting tube 48 and handle 46 to position insertion tube 17 inside overtube 10, as shown in FIG. 6. Once in place, elongated member 32 is pulled taut to close loops 44 and securely close slot 26 around insertion tube 17. This and similar pre-threaded configurations should reduce the amount of time and the cumbersomeness of having to thread elongated member 32 through holes 30 when overtube 10 is needed to complete the endoscopic procedure.

In use, when the advancement of insertion tube 17 is prevented by the formation of a sigmoid loop, looping in the stomach or other reasons, the operator or physician will pull a portion of insertion tube 17 out of the patient to reduce the sigmoid loop and then to place overtube 10 around insertion tube 17. With slot 26, overtube 10 is loaded by placing endoscope lumen 16 around insertion tube 17 in a side-to-side fashion through slot 26. Once overtube 10 is loaded, elongated member 32 is threaded through holes 30 and then pulled tight to securely close slot 26. Generally, it will be preferred for the operator, or one of his or her assistants, to first close slot 26 manually by closing his or her hand around overtube 10 in order to facilitate threading of elongated member 32 through holes 30. Once elongated member 32 is pulled taut and slot 26 securely closed, such that knot member 36 is pulled against the first of threaded holes 30, at least the leading end 42 of elongated member 32 is provided with a fastener member 40, which can simply be a knot at the end 42 or a separate member to maintain elongated member 32 taut, and slot 26 securely closed. Besides preventing elongated member 32 from being pulled through holes 30, knot member 36 provides a mechanism by which elongated member 32 can be pulled taut.

While there are shown and described herein certain specific alternative forms of the invention, it will be readily apparent to those skilled in the art that the invention is not so limited, but is susceptible to various modifications and rearrangements in design and materials without departing from the spirit and scope of the invention. In particular, while the above description contains many specifics, these should not be construed as limitations on the scope of the invention, but rather as an exemplification of one or more preferred embodiments thereof. Further, it should be noted that the present invention is subject to modification with regard to assembly, materials, size, shape and use. For instance, some of the components described above can be made integral with each other to reduce the number of separate components.

What is claimed is:

1. An overtube for use with an endoscope, comprising:
a generally elongated tubular member having a proximal end, a distal end and an outer surface, said elongated tubular member configured with a cross-section having a thickened portion;
an endoscope lumen disposed in said tubular member, said endoscope lumen sized and configured to slidably receive the endoscope;
a slot disposed along said tubular member through said thickened portion, said slot interconnecting said endoscope lumen with said outer surface of said tubular member for receiving the endoscope into said endoscope lumen through said slot,
a plurality of holes in said thickened portion through said slot; and
an elongated member threadably received in said plurality of holes to close said slot and maintain said slot in a closed position, said elongated member having at least a leading end.

2. The overtube of claim 1, wherein said thickened portion is generally parallel to said endoscopic lumen.

3. The overtube of claim 1, wherein said elongated member has a trailing end, said leading end configured to fit through said plurality of holes, said trailing end having a knot member to prevent passage of said trailing end through said plurality of holes.

4. The overtube of claim 3 further comprising a fastener member at said leading end of said elongated member to maintain said slot in said securely closed position.

5. The overtube of claim 1, wherein said elongated member comprises a plurality of loops, said plurality of loops configured to fit over a handle and a connecting tube of the endoscope.

6. The overtube of claim 1, wherein said plurality of holes are generally transverse relative to said thickened portion and said slot.

7. The overtube of claim 1, wherein said plurality of holes are angulated relative to said thickened portion and said slot.

8. The overtube of claim 1, wherein each of said plurality of holes has at least one hole generally aligned therewith across said slot.

9. A method of performing a gastrointestinal endoscopy procedure in a gastrointestinal tract using an endoscope and an overtube, said overtube having a longitudinal slot, said method comprising the steps of:
a) introducing said endoscope into said gastrointestinal tract;
b) inserting said endoscope into said gastrointestinal tract until a loop is formed in said gastrointestinal tract or said endoscope;
c) withdrawing said endoscope as necessary so as to straighten said loop in said gastrointestinal tract or said endoscope;
d) loading said overtube onto said endoscope by placing said endoscope through said longitudinal slot into a endoscopic lumen in said endoscope;
e) threading an elongated member through a plurality of holes disposed in a thickened portion of said overtube by crossing said slot with said elongated member;
f) pulling said elongated member taut to close said slot;
g) inserting said overtube into said gastrointestinal tract; and
h) completing said endoscopy procedure.

10. The method of claim 9, wherein said thickened portion is generally parallel to said endoscopic lumen.

11. The method of claim 9, wherein said plurality of holes are generally transverse relative to said thickened portion and said slot.

12. The method of claim 9, wherein said plurality of holes are angulated relative to said thickened portion and said slot.

13. The method of claim 9, wherein each of said plurality of holes has at least one hole generally aligned therewith across said slot.

14. A method of performing a gastrointestinal endoscopy procedure in a gastrointestinal tract using an endoscope and an overtube, said overtube having a longitudinal slot with an elongated member threaded through a plurality of holes disposed in said overtube, said elongated member forming a plurality of loops, said method comprising the steps of:
a) introducing said endoscope into said gastrointestinal tract;
b) inserting said endoscope into said gastrointestinal tract until a loop is formed in said gastrointestinal tract or said endoscope;
c) withdrawing said endoscope as necessary so as to straighten said loop in said gastrointestinal tract or said endoscope;
d) separating a connecting tube of said endoscope from a light source, a suction tube and a water tube;
e) loading said overtube onto said endoscope by placing said loops over said connecting tube, said handle and an insertion tube and placing said insertion tube through said longitudinal slot into an endoscopic lumen in said overtube;
f) pulling said elongated member taut to close said slot;
g) inserting said overtube into said gastrointestinal tract; and
h) completing said endoscopy procedure.

15. The method of claim 14, wherein said plurality of holes are located in a thickened portion of said endoscope.

16. The method of claim 15, wherein said thickened portion is generally parallel to said endoscopic lumen.

17. The method of claim 14, wherein said plurality of holes are generally transverse relative to said thickened portion and said slot.

18. The method of claim 14, wherein said plurality of holes are angulated relative to said thickened portion and said slot.

19. The method of claim 14, wherein each of said plurality of holes has at least one hole generally aligned therewith across said slot.
